(19) United States
(12) Patent Application Publication
(10) Pub. No.: US 2025/0037831 A1
(43) Pub. Date: Jan. 30, 2025

(54) Methods and Systems for Monitoring and Delivery of Therapeutic Medicines

(71) Applicant: SHL Medical AG, Zug (CH)

(72) Inventors: Katherine Ford Brigham, Rutherford, NJ (US); Madeleine Hayden Anderson, Union City, NJ (US); Amy Catherine Rinaldi, Morristown, NJ (US); Matthew Conner D'Auria, Bernardsville, NJ (US); James Thomas Hawthorne, Milbury, MA (US); Demosthenis Katsaros, Walpole, MA (US); Martin Michael Coyne, III, Towaco, NJ (US)

(21) Appl. No.: 18/782,245

(22) Filed: Jul. 24, 2024

Related U.S. Application Data
(60) Provisional application No. 63/530,748, filed on Aug. 4, 2023.

(30) Foreign Application Priority Data
Jul. 25, 2023 (EP) 23386067.5

(51) Int. Cl. G16H 20/17; G16H 50/30

(52) U.S. Cl. CPC G16H 20/17; G16H 50/30

(57) ABSTRACT

The present disclosure provides a method of providing visual feedback during a medicament injection to a patient. The method includes receiving, via a first computing device, information regarding the medicament injection. The method also includes displaying, via a display of a second computing device, a name of the medicament and a dosage amount of the medicament for the medicament injection. The method also includes displaying, via the display of the second computing device, one or more setup steps for preparing the medicament injection. The method also includes displaying, via the display of the second computing device, an injection dashboard providing one or more indications of a progress of the medicament injection.

### CROSS-REFERENCE TO RELATED APPLICATIONS

The present application claims priority to (i) European Patent Application No. 23386067.5 filed Jul. 25, 2023, and (ii) U.S. Provisional Patent Application No. 63/530,748 filed Aug. 4, 2023. The entire disclosure contents of these applications are herewith incorporated by reference into the present application.

### TECHNICAL FIELD

The present disclosure generally relates to apparatus, systems, and methods for using functionalities of common household devices to improve monitoring and delivery of therapeutic medicines.

### BACKGROUND

Autoinjectors (AIs) and on-body injectors (OBIs) have relatively short injection times, and a predictable start and end of dose that is on the order of seconds to minutes. Existing digital patient support systems for self-injection are tightly coupled to the devices they were designed for; a single device with a single medication and a short injection duration.

However, not all patients are candidates to receive therapy with a single AI or OBI device. Some patients require complex, ongoing treatment to manage their conditions. Such treatments may include individual medications or multi-medication regimens given by one or more routes of administration. Medications, medication regimens, and routes of administration generally correspond to a specific disease state and treatment regimen and may be customized to a specific patient's clinical needs, disease progression, or physical and/or cognitive limitations. Medication regimens may also change during the course of therapy, as with oncology regimens. The devices and complex regimens may be overwhelming for patients at home who lack clinical training, have low literacy or health literacy, or suffer limitations from underlying disease.

Many drug delivery devices used to deliver treatments are disposable, single-use devices that are mass-produced. Practical cost and manufacturing constraints limit inclusion of advanced functionality that would improve intuitiveness and patient usability. As a result, many drug delivery devices are limited to simple interaction elements including pushbuttons, mechanical audible/tactile feedback mechanisms, or moving visual indicators. There is a need for improved methods of feedback and more intuitive user interactions that would be prohibitive to implement on lower-cost, disposable devices themselves. These needs are particularly relevant for delivery of more complex therapies, such as multi-medication regimens, especially when self-administered at home by a patient or user lacking clinical training, such as a caregiver, family member, or friend.

Additionally, certain medications have a higher relative propensity to induce sudden, unpredictable systemic reactions related to the origin, pharmaceutics, or mode of action of the medication upon the body. While rare, systemic reactions are potentially fatal, demanding immediate halt to medication administration, and emergency administration of one or more counteracting medications. Clinicians are skilled at identifying and responding to systemic reactions. However, in the home setting in the absence of a trained clinician, patients must rely on themselves, a caregiver (e.g., a family member), or emergency responders for intervention. As such, there is a need for drug delivery devices that enable safe home use by patients themselves or lay (non-clinical) caregivers, enabling effective emergency treatment if needed.

Further, there is a need to provide information to an emergency responder in the home if a reaction takes place and emergency services are summoned by the patient. The information required by an emergency responder is far more detailed and clinically-based compared to the information presented to the patient. Ideally, home-based delivery devices would have the capability to change the information, tone, and content based on the context of use (e.g., normal delivery with a patient in simple language, or complex clinical “shorthand” in an emergency).

### SUMMARY

Principles and embodiments of the present disclosure relate to systems and methods for using functionalities of common household devices, such as Internet-enabled televisions (e.g., smart TVs) or other streaming devices, to improve monitoring and delivery of therapeutic medicines. More particularly, the present disclosure is directed towards more intuitive approaches to monitor and control drug delivery systems, especially those delivering more complicated medication regimens or medications that may cause a patient to suffer a reaction, such as a systemic infusion reaction. Further, the present disclosure is directed towards providing timely, effective emergency care for situations that may necessitate medical attention from a trained provider should a systemic infusion reaction occur.

The apparatus enables a user to configure, monitor, and/or control a drug delivery apparatus before, during, and after administration of medications. A smart TV or other streaming unit present in a patient's home is used as pre-existing hardware, upon which a software specific to the drug delivery device is installed. The software application, once installed, configured, and coupled to a drug delivery system, may interrogate and/or control one or more aspects of the coupled drug delivery device or display computer-generated status data in understandable terms to the user, all as described below. Patients will likely be near their TV or streaming unit, particularly to pass time during longer infusions. By using familiar household devices and interaction patterns, even complex therapy is easily understandable.

Complexity of the actual drug delivery device is also reduced, as the processing of user requests and responses is handled by the software application already provided in the smart TV or streaming unit. These are particularly advantageous for users lacking clinical training (such as the patient themselves or a lay caregiver or family member), those with cognitive or physical limitations, or for complex therapies.

As such, the present disclosure provides a method of providing visual feedback during a medicament injection to a patient. The method includes receiving, via a first computing device, information regarding the medicament injection. The method also includes displaying, via a display of a second computing device, a name of the medicament and a dosage amount of the medicament for the medicament injection. The method also includes displaying, via the display of the second computing device, one or more setup steps for preparing the medicament injection. The method also includes displaying, via the display of the second computing device, an injection dashboard providing one or more indications of a progress of the medicament injection.

These as well as other aspects, advantages, and alternatives, will become apparent to those of ordinary skill in the art by reading the following detailed description, with reference where appropriate to the accompanying drawings.

### BRIEF DESCRIPTION OF THE FIGURES

FIGS. 1A-1D illustrate schematic drawings showing the interactions between the various devices throughout the full process of the present disclosure, according to an example embodiment.

FIG. 2A-2C illustrate schematic drawings showing a tone flow logic, according to an example embodiment.

FIG. 3A-C illustrate schematic drawings showing a process for verifying the medication and device information, according to an example embodiment.

FIG. 4 illustrates an example display screen during a device confirmation step, according to an example embodiment.

FIG. 5 illustrates an example display screen during a setup step, according to an example embodiment.

FIGS. 6A-6D illustrate a schematic drawing of a dashboard view that provides guidance to the patient through the active phases of a drug infusion, according to an example embodiment.

FIGS. 7A illustrates an example display screen during an infusion step, according to an example embodiment.

FIG. 7B illustrates a plurality of example display screens on a smart watch, according to an example embodiment.

FIGS. 8A-8D illustrate schematic drawings of a dashboard view showing the different triggers for an emergency state, according to an example embodiment.

FIG. 9 illustrates an example display screen during an infusion step, according to an example embodiment.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

In the Figures, the blocks may represent operations and/or portions thereof and lines connecting the various blocks do not imply any particular order or dependency of the operations or portions thereof. It will be understood that not all dependencies among the various disclosed operations are necessarily represented. The Figures and the accompanying disclosure describing the operations of the method(s) set forth herein should not be interpreted as necessarily determining a sequence in which the operations are to be performed. Rather, although one illustrative order is indicated, it is to be understood that the sequence of the operations may be modified when appropriate. Accordingly, certain operations may be performed in a different order or simultaneously. Additionally, those skilled in the art will appreciate that not all operations described need be performed.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, apparatus, element and method “configured to” perform a specified function is indeed capable of performing the specified function without any alteration, rather than merely having potential to perform the specified function after further modification. In other words, the apparatus, element, and method “configured to” perform a specified function is specifically selected, created, implemented, utilized, programmed, and/or designed for the purpose of performing the specified function. As used herein, “configured to” refers to existing characteristics of an apparatus, element, and method which enable the apparatus, element, and method to perform the specified function without further modification. For purposes of this disclosure, an apparatus, element, and method described as being “configured to” perform a particular function can additionally or alternatively be described as being “adapted to” and/or as being “operative to” perform that function.

### Embodiment 1: Device Ecosystem

The present disclosure forms part of an ecosystem that uses familiar interaction patterns and device interfaces to guide users through their infusion and make even complex therapy understandable. The “digital caregiver” described herein is a verification, setup, and monitoring tool to guide patients through an at-home infusion. The patient can track their infusion process from the point that they receive their medication through to the completion of their infusion.

Once the infusion starts, the software application can display real-time infusion progress. This is useful if the patient is watching TV, or the infusion has a longer duration. Users can also ask a linked voice assistant questions and receive a response in simple, easy-to-understand terms. The software application presents information about the infusion to the user while hiding the complexity of the underlying device and regimen to prevent confusion and information overwhelm. If the patient is moving around their house they can check their progress using their smartwatch. This setup allows the software application to be accessible when the patients are either sedentary or mobile.

During the infusion, the patient can monitor the system for a potential infusion reaction using the software application or a smartwatch. If an infusion reaction occurs, emergency services will likely be summoned to the home. To prepare an emergency responder, the system switches to a “clinical” mode that rapidly provides information in language familiar to a healthcare provider. The goal is to aid emergency workers with high-salience (and potentially real time or near-real-time) information to brief them on the patient's infusion reaction.

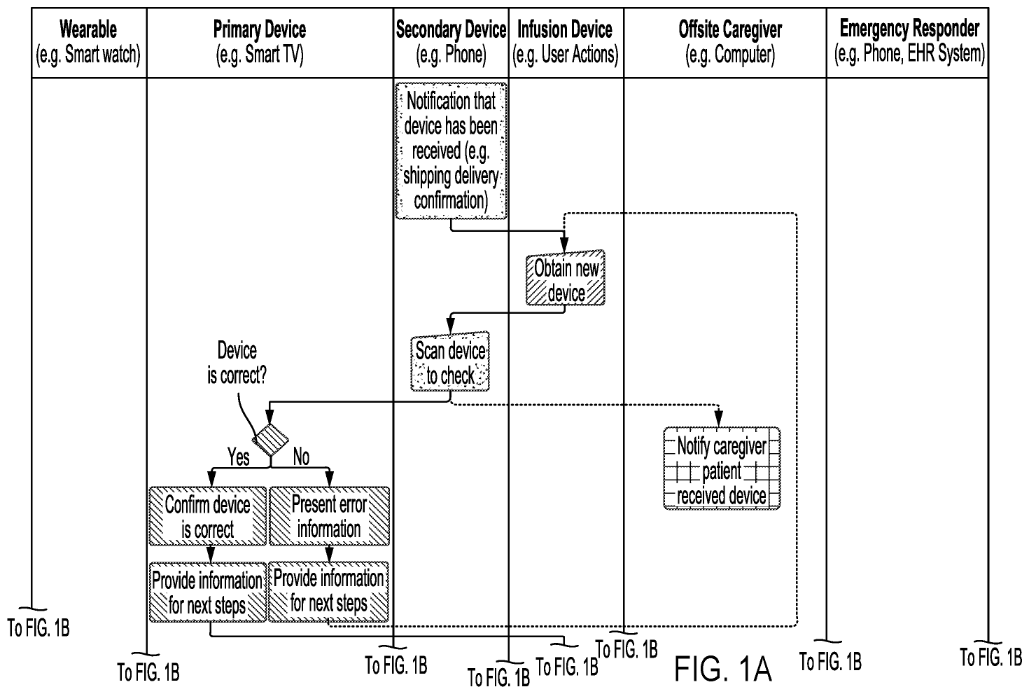

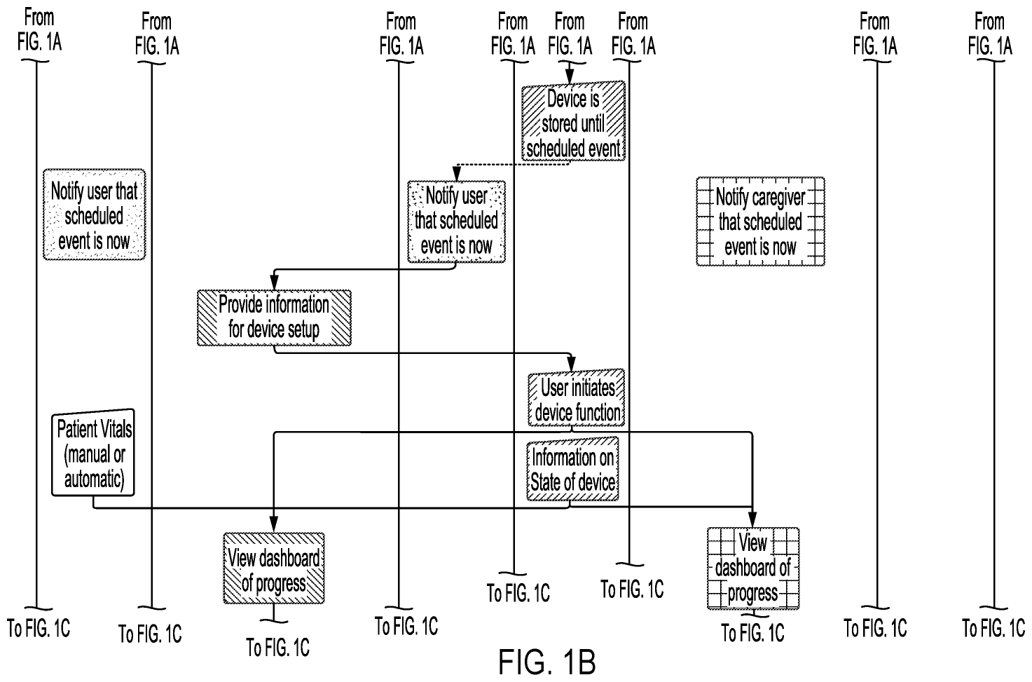

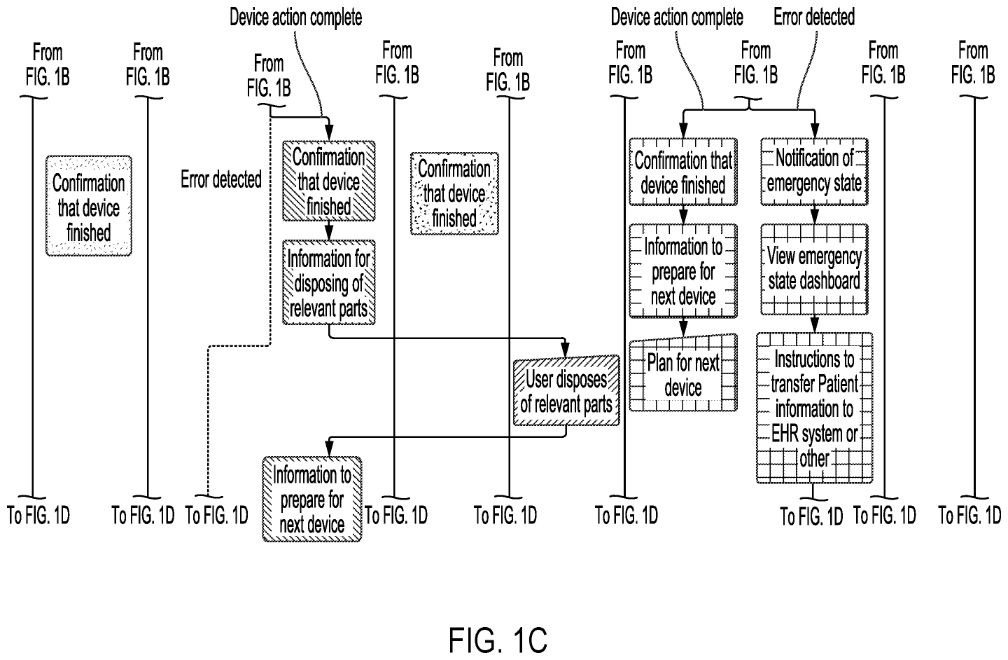

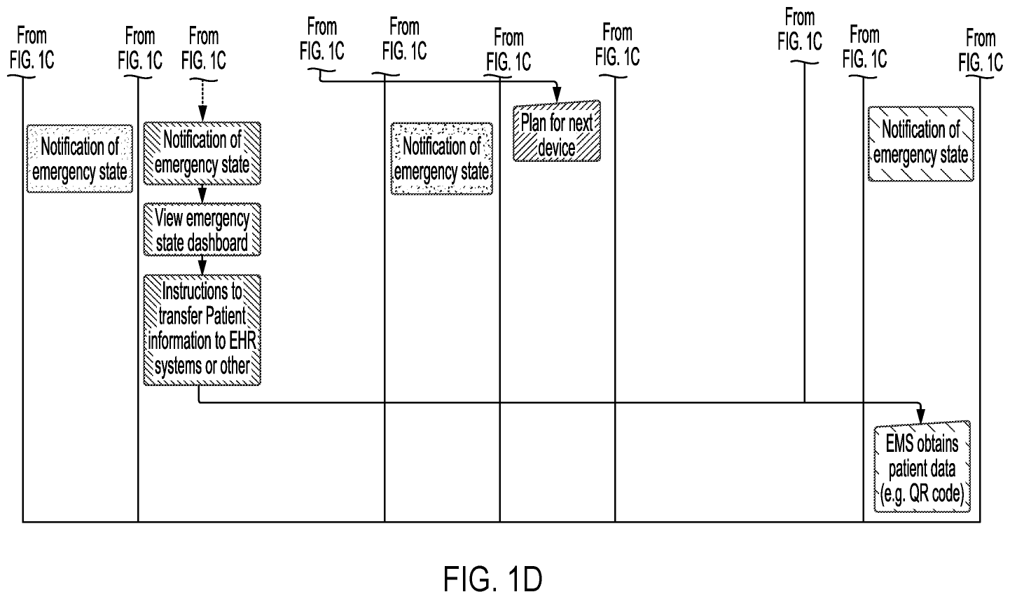

The digital caregiver incorporates a varying range of device types to accommodate a range of patient scenarios. FIGS. 1A-1D shows the interactions between the various devices throughout the full process of using the present invention. The system does not require all the devices to operate but can leverage what is available to expand features and enhance functionality. The TV serves as the primary display for the digital caregiver interface, and the phone and smartwatch are peripheral devices that can extend the reach of the digital caregiver throughout the home. As the user moves about their house and goes about their day, they can stay up to date on the status of their infusion.

Wearable devices (such a smartwatch or other Internet-enabled watch as non-limiting examples) can be leveraged as both a notification system and to provide inputs to the system. The user may be notified through their wearable of a scheduled event, the successful completion of an event, or an error with the process. Devices like a smartwatch also offer data on a user's vitals, and when worn by the patient can be used to obtain live health data relevant to the event.

Primary Device (e.g. Smart TV)—The present disclosure utilizes a television interface as the central interaction point. This is driven by the primary location of use, the home. The Smart TV offers an increased screen size. The background can be linked to the user's personal smart TV screen background to personalize the viewing experience and create a more familiar layout.

Secondary Device (e.g., Smartphone or Smartwatch)—The secondary device offers a mobile interface for notifications and simple interactions. A smartphone adds camera functionality and may be used to scan QR codes on the medical device. A smartwatch or other wearable devices may provide the patient's vitals to monitor the patient for a potential infusion reaction, as discussed in additional detail below.

Medical Device (e.g., Infusion Device)—Feedback and data from the medical device provide the necessary data to inform the user about the infusion progress. The digital caregiver may also have the ability to interrogate the medical device for specific data, and may be able to control the infusion device.

Caregiver Device (e.g. Computer Offsite)—A caregiver notification system and dashboard may be used to expand the reach of health care professionals (“HCPs”) to provide offsite support and monitoring of at-home infusions. HCPs could manage multiple patient infusions simultaneously from a remote monitoring site, and be on-call for assistance or guidance if required.

Emergency Responder Device (e.g. Phone, EHR System)—In the case of an adverse reaction or emergency, the digital caregiver can provide information on the patient and infusion status to an emergency responder. The information may be obtained by manual documentation by emergency medical services (“EMS”), through scanning a QR with a phone, or through direct integration with an electronic health record (“EHR”) system.

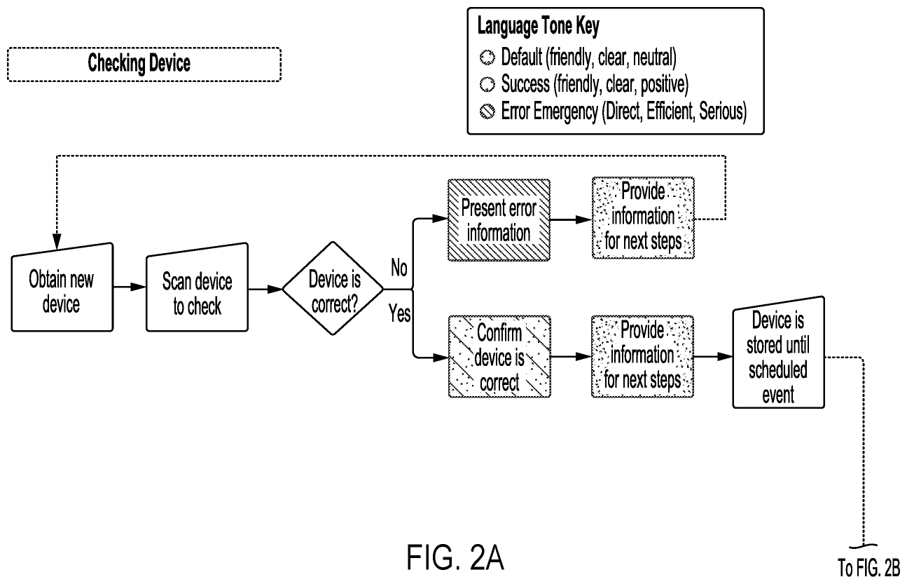

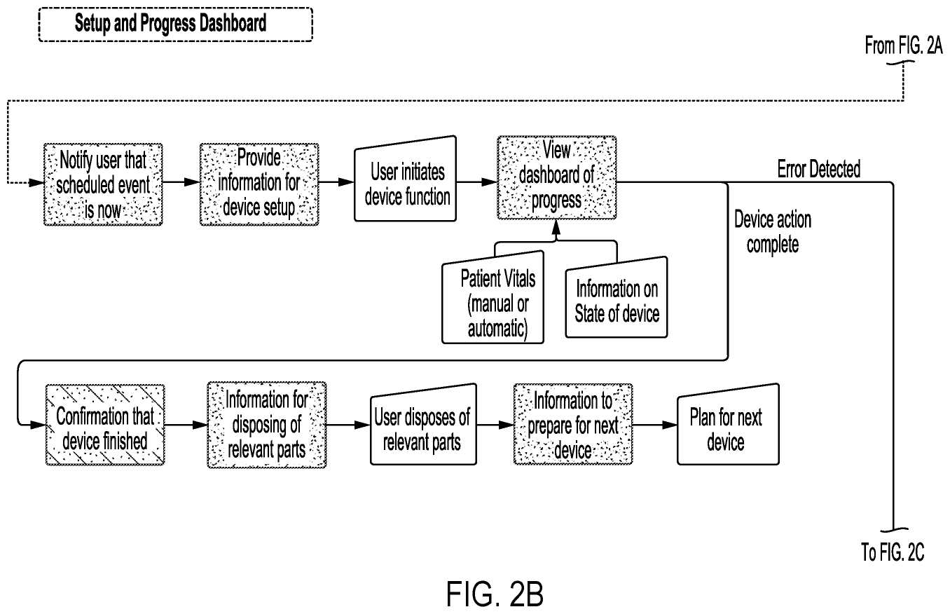

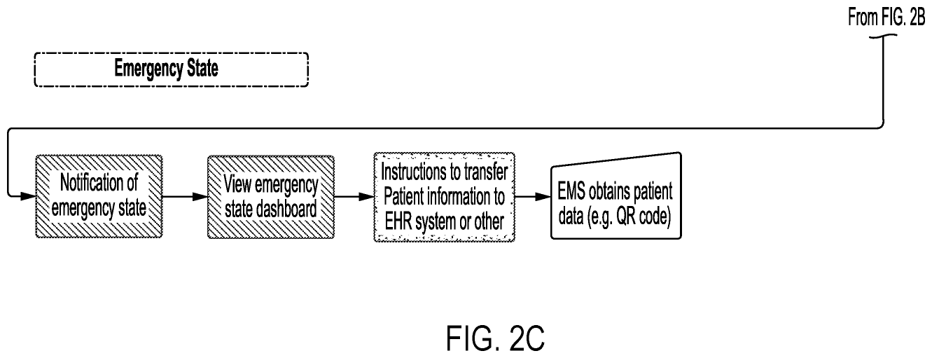

The user base for the digital caregiver varies by context and is designed for users with varying health literacy. In order to meet the needs of a wide range of users, the digital caregiver utilizes a lower reading level to keep the content clear and easy to interpret. The content tone conveys context for how the information should be interpreted by the user. In the case of a successful action, a positive tone reinforces the feedback. If there is an error or issue then a shift in tone may communicate the severity of the situation. In the case of an emergency situation, the use of clear succinct language may improve the speed of information transfer to EMS and the patient outcome. FIGS. 2A-2C are a schematic drawings showing tone flow logic, according to an example embodiment.

### Embodiment 2: Verification of Medication and Materials

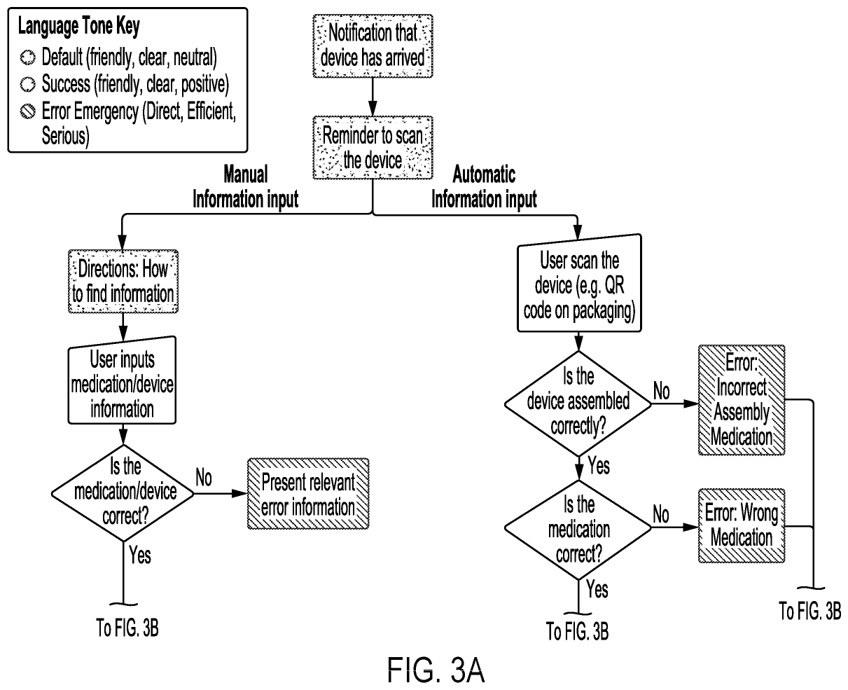

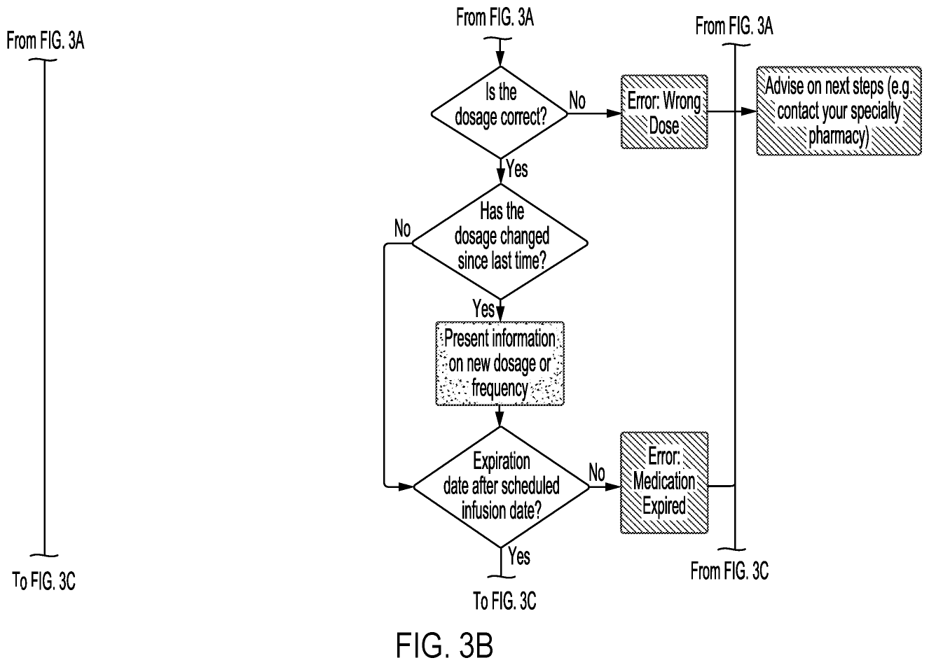

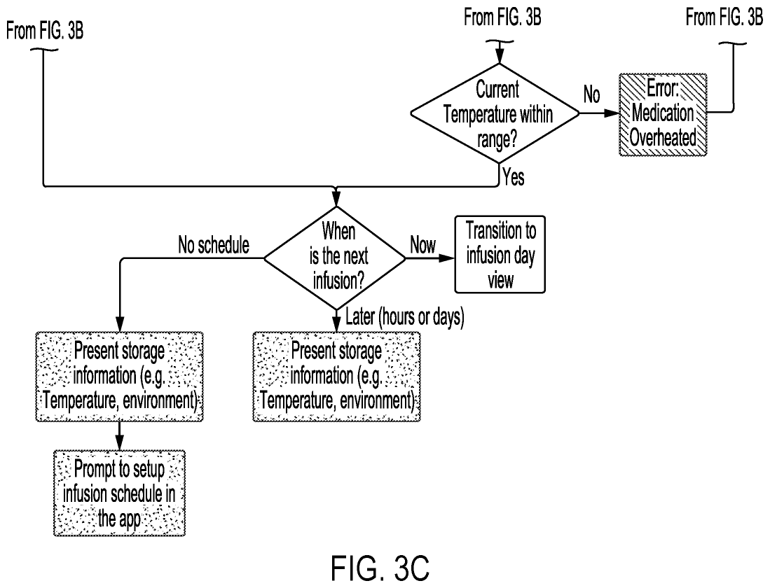

The user can scan their medication when it arrives using the camera on their phone and view the content of the infusion device. This may include information such as the medications and the programmed infusion rates for the device. This information can be automatically verified with the patient's prescription information that is stored in the digital caregiver's patient profile. Having the patient scan the medication when it first arrives allows enough time to fix any dispensing errors before the infusion day. The patient can review their medication regimen and confirm that it was dispensed correctly. FIGS. 3A-3C shows a process for verifying the medication and device information.

The present disclosure anticipates potential points of friction for the user and provides tools to avoid errors. When the patient receives their device and/or medication, they may scan the information into the digital caregiver to confirm that everything is correct. If any errors are identified, it can provide the user with actionable steps to deal with the situation. This may include obtaining a new dose of medication from their specialty pharmacy, updating their prescription information in their patient profile, or learning about proper storage and infusion techniques. The method by which the digital caregiver obtains information about the medical device and medication may be either manually by a patient or caregiver or automatic through the use of a QR code or other touchless technology.

The error feedback provided to the user may be tailored to the subject of the error, and the severity of the effect it will have on the infusion. Most errors will result in the patient order new medication. After checking the medication and device, the patient will need to store their medication (e.g. in the refrigerator) until their infusion day. This is a salient moment to provide instruction on how to store the device as they are going to take immediate action with the information. If today is the infusion day this may be the appropriate time to transition the user into the setup and dashboard view for their infusion. If there are changes to the patient's regimen (e.g. new medication, different dosage, different use steps) the digital caregiver may provide information on how the patient's regimen has changed, what actions they may need to take, and the reason for the change in regimen.

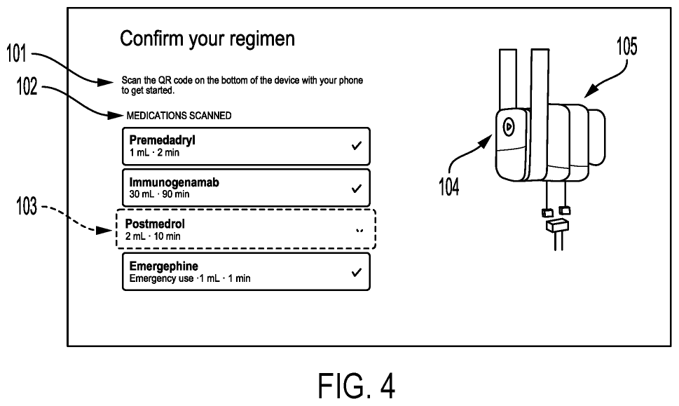

Referring to FIG. 4 which illustrates an exemplary smart TV device confirmation screen, Instructions are provided to the user 101 on how to scan the information on their device. This may be in the form of a QR code or through a Bluetooth connection between the digital caregiver software application and the infusion device. Further information may be provided that is dependent on the technological proficiency of the target user. During this stage, a peripheral phone software application could be used to act as the “eyes and ears” of the smart TV app. This is especially relevant when taking actions like scanning a QR code or connecting via Bluetooth.

The software application may provide feedback to the user that the medications have been successfully scanned, as seen in 102. In the case of an error with the device, the user may be presented with the information that is incorrect and provided with the opportunity to either update their prescription information if it is the software application that is out of date, or they may be advised to contact their healthcare provider or pharmacist to receive the correct device for their infusion. There may be a secondary manual check, if the user needs to scroll through, as shown in 103, to check the medication information that was received from the device.

A depiction of the device 104 may be shown for visual reference and may be used as a tool to communicate progress to the user. The information received from the device may populate incrementally, so the device image can act as a modified progress bar with an emphasis on each section 105 as the information is received and processed. This visual emphasis on each section may also be manually triggered as the user scrolls through the different medications. The visual of each highlighted section of the device will help acquaint the user with how their medication is organized or stored within the device.

The medication confirmation interface can be used to communicate any changes in the treatment regimen to the patient. An example of this is treatment plans with a variable dosing scheme. The prescription information stored in the software application may be updated by either a patient, caregiver, or HCP, so this interface offers the opportunity for HCPs to communicate any changes to their patient. HCP access to the patient's prescription settings, would help ensure the accuracy of the interface, and give the patient peace of mind that they have the correct settings in their digital caregiver user profile.

The user may scan each medication separately or scan the entire device at once depending on how the information is coded in the device. Once the device is verified as correct, there is the opportunity to guide the user on the next step of their treatment. This is a salient moment to give instructions on storage methods for the device between the point that they have received and scanned the device, and when they reach their infusion day.

### Embodiment 3: Visualization of Setup

A setup flow can be used to guide the user through preparing for their infusion. Many patients may spend the time during their infusions on the couch in front of their TV. The TV offers a large screen to present setup information that may otherwise be found in small print or paper format packaged with their device. The setup process also initiates their interaction with the TV software application interface for the remainder of their infusion and will lead in smoothly to their infusion dashboard Embodiment 4.

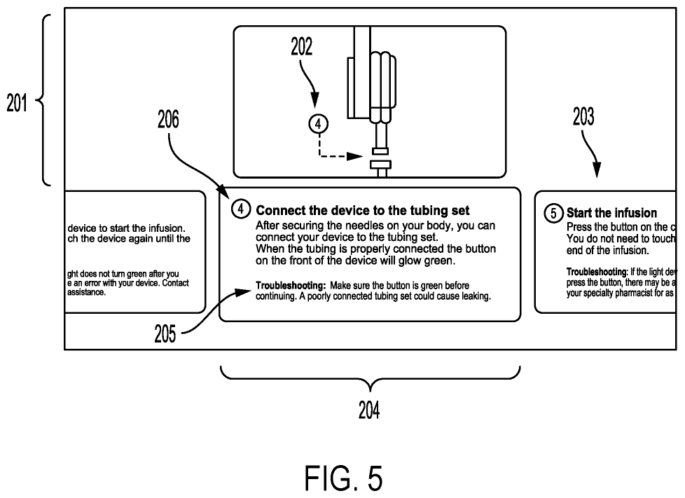

Referring to FIG. 5 which illustrates an example of a TV view of the setup screen, a series of steps can be presented to the user. In this example, the user can scroll horizontally to navigate backward and forward through the steps so that they can proceed at their own pace. As the user navigates through the steps, they may be able to see a preview of the future step 203 to orient themselves. This may offer comfort and confirmation for the user of where they are in the process and give them a warning of what is to come.

The information for each step may be centrally located 204 to focus the user's attention. For each step, the user may see both written explanations and a visual depiction 201. The visual of the device may contain a specific callout 202 of the relevant parts of the device or it may contain an illustration or animation of the step. The written explanation may consist of the main body of text about the actions that need to be taken 206 and then supplemental troubleshooting information to proactively address any issue or confusion the user may have 205. The troubleshooting information 205 can aid users that know they are having a problem and proactively assist users that didn't realize they were making a mistake. This can also prevent irreversible errors before they are made, which is particularly important for expensive medication or devices with long lead times for delivery.

Automatic feedback between the device and the digital caregiver offers the opportunity to update the instructions with the user's progress. When the device knows a user has completed a step, the instructions can update to show the user's progress and where they are in the process. Using callouts on the static illustration can bring the user's attention to which portion of the device they should be focusing on for a given setup step. The digital image of the device can grow and change as the user assembles the device to reflect their progress. This automatic update can offer feedback that the user is assembling the device properly.

The larger television screen can also optionally display the full set of setup steps at once while maintaining a legible text size. With a static view, there is no need to physically interact with any controls during the setup phase. This is specifically beneficial for steps that require clean hands, like cleaning the injection site or inserting any needles. The user does not need to touch anything unsanitary like their phone or the TV remote. The user will also have a full view of the process and will know what is coming next which can remove uncertainty about where they are in the process. The TV format and screen size are first and foremost designed for viewing video content on a large scale, so it seamlessly allows for any instructional videos to supplement the other digital caregiver instructional software application content.

The digital caregiver may also incorporate communication with EHR systems to allow for integration with information like lab results. This patient data may be used to confirm in real-time necessary lab results that gate the infusion process. It may also be used to alert users to missing lab information that will need to be resolved before their infusion day.

Once the user has confirmed the device is set up and ready, they can start the infusion. This may shift the software application to Embodiment 4 and the infusion will begin.

### Embodiment 4: Progress Dashboard

Patients are performing their infusions at home, and may not have an HCP or caregiver with them. The dashboard view is the core functionality of the digital caregiver and provides guidance through the active phases of the infusion. The digital caregiver may also provide information and infusion status to offsite assistance (e.g. nurse or family member that is not at home) and offer tools for the patient to communicate with their support system. The dashboard provides live feedback on the infusion process and translates the status of the infusion device and the information it holds into clear actionable feedback for the user. The dashboard may also guide patients and their caregivers through any errors or issues they have with the device (e.g. issues during setup of the device, errors with the device during infusion, or any adverse reactions to the medications). The dashboard has the primary state when the infusion is progressing as planned, and a secondary emergency state triggered by an adverse reaction from the patient (the emergency state dashboard is addressed in Embodiment 5).

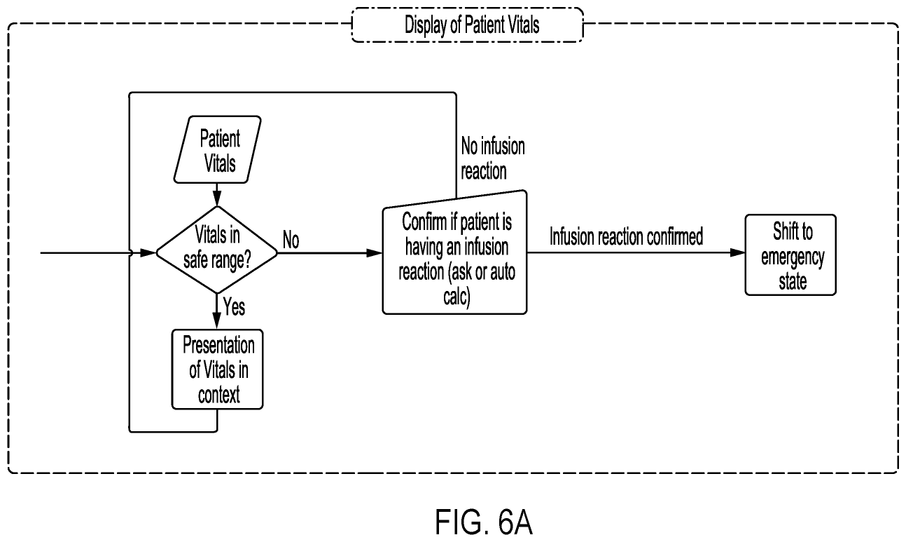

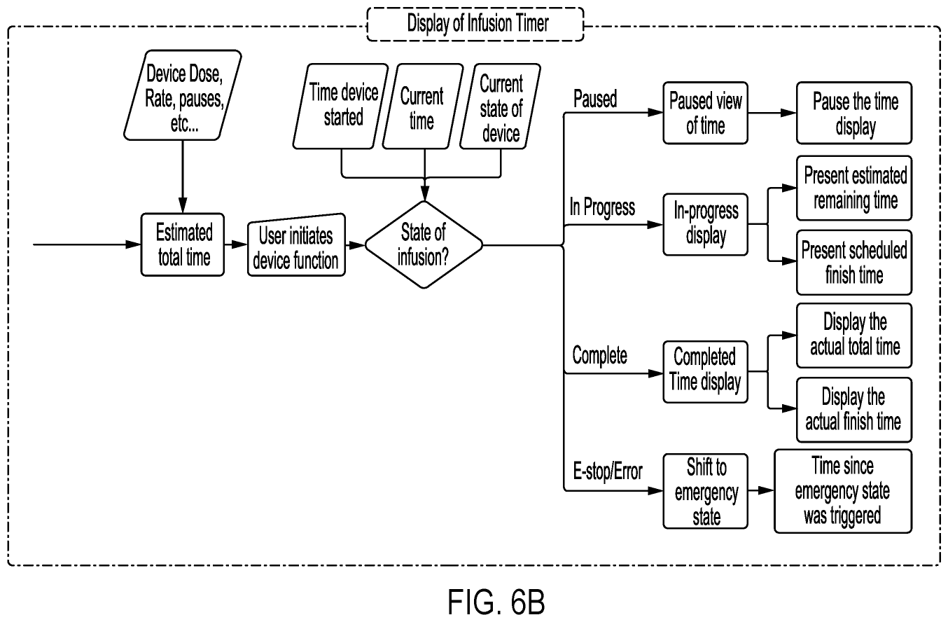

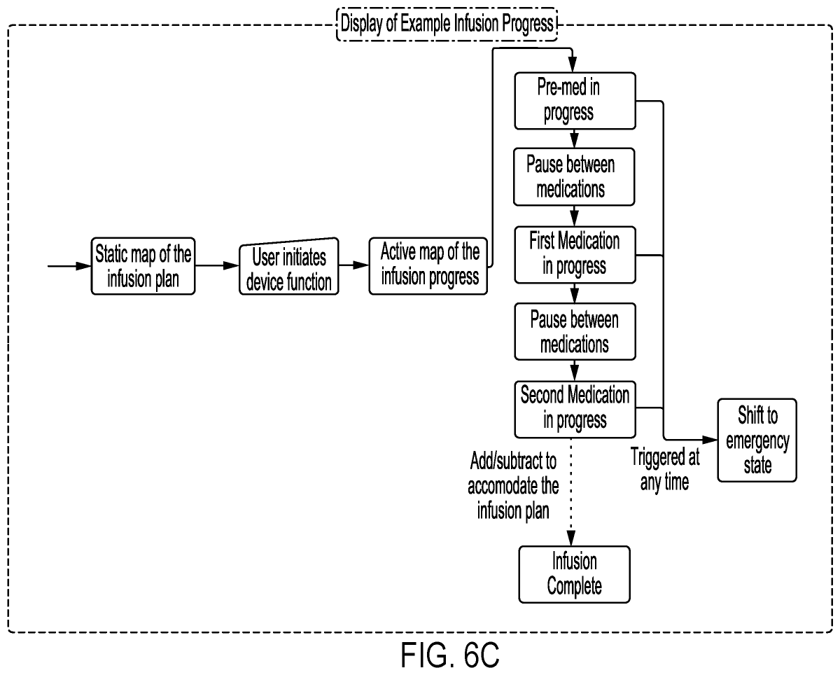

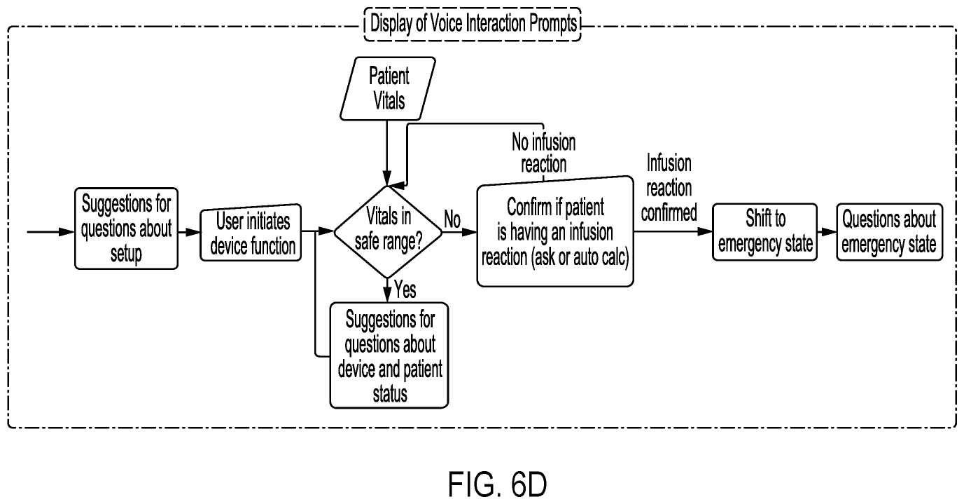

Patient Vitals (FIG. 6A)—Obtained from a wearable device on the patient, the vitals may be presented on the dashboard to have them visible in the context of the infusion that is happening. The vitals may provide information on the patient's health throughout the infusion and be used as a metric for identifying an infusion reaction. The threshold of “healthy” vitals that would trigger an event may be set by a patient, HCP, or from known standards.

Infusion Timer (FIG. 6B)—The timer provides a prospective look at the remaining infusion time that may be estimated based on the dosage and flow rate information from the infusion device. This information may include the estimated end time of the full infusion so that the user does not need to calculate it themselves, and so that the patient can plan their day around the infusion.

Infusion Progress (FIG. 6C)—The infusion progress may also be mapped out visually to provide information on each stage of the infusion, since multiple medications and long periods of time may be involved.

Voice Interaction Prompts (FIG. 6D)—Users may be able to interact with a voice assistant that is either standalone or the native voice assistant in their device. The user may be presented with suggested prompts to assist them in learning what information is available. These prompts match the stage of the process that the user is in (e.g., in setup there may be troubleshooting questions presented, while in the emergency state there may be questions about the patient's medical state presented to the user).

With the setup process on the TV, the user can then seamlessly transition to a TV dashboard view of their progress without having to change interfaces. A dashboard view keeps the user up to date on the progress of their infusion and offers an opportunity to prompt further interaction with other aspects of the digital caregiver. Suggestions for voice prompts can be presented visually to the user to remind them of the different ways they can communicate with the digital caregiver.

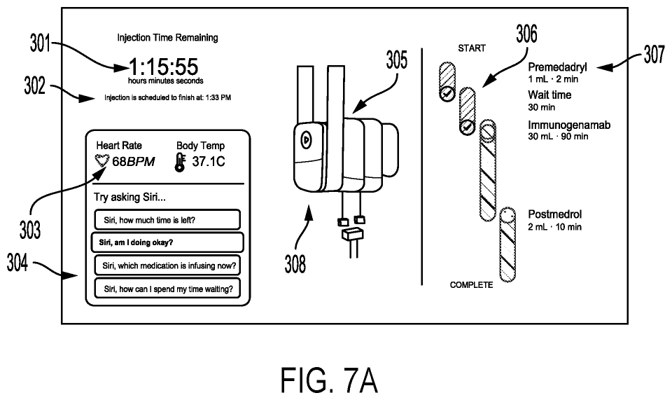

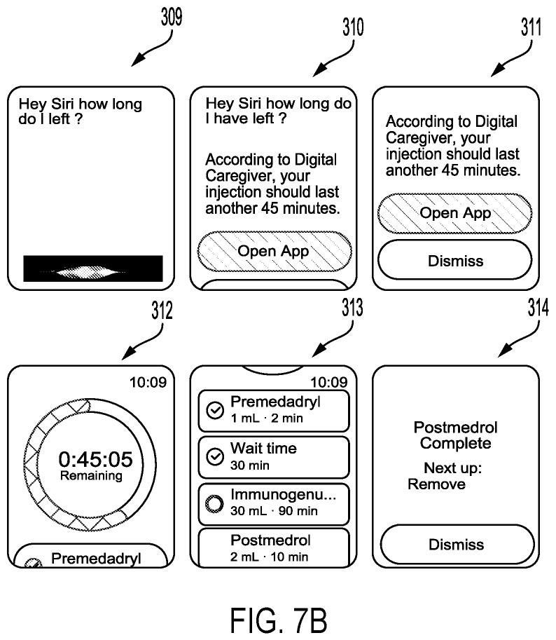

Referring to FIG. 7A illustrating the television dashboard view for a patient during the infusion process, the overall remaining injection time 301 can be presented to the user with a scheduled finish time 302 to help them plan their day. The vitals 303 may be tracked and presented to the user to keep them up to date on their body during the infusion. The vitals may be presented with color coding and icons to indicate whether they are in-range or out of range. If the various vitals are outside of a healthy range the digital caregiver may use that information to determine if the patient is having an infusion reaction. Abnormal vitals may be used to trigger a prompt to ask the user how they are feeling and if they are having an infusion reaction.

The present disclosure also utilizes a voice interface to allow the user to ask questions and troubleshoot or interrogate the infusion device. Voice systems rely on users knowing and remembering that they are there for them to interact with. The suggested voice prompts 304 may offer a visual queue to initiate interaction with the voice assistant system. The user may be able to scroll through multiple suggested prompts 304 that could be organized by subject matter or urgency of the issue. The voice prompts may automatically scroll through slowly to give a rolling carousel of suggestions to the user.

The dashboard may offer multiple methods of displaying the infusion progress to the user. There may be a timeline of the infusion with labeled portions showing the sequence of medications 307 and abbreviated information about each stage of the infusion including dosage and approximate time. Each section may have a certain visual state 306 determined by the progress of that portion of the injection. With the use of icons and animations, the sections can slowly fill up at the pace of the injection and then display a completed state.

Visual representation of the device 308 on the dashboard can be used to help the users orient themselves with the progress of the device. This may be a photograph of the device or a realistic depiction or illustration that offers clear visual references that allow the user to recognize the device as their own specific device through unique visual elements and real-time feedback. The user could customize the view using visual elements like color or pattern to personalize the feel of the digital dashboard. The real-time feedback 305 can be visually displayed on the depiction of the device, emphasizing sections of the device when they are in use, to show the user what part of their device is currently active and help them gain a greater understanding of how their device works.

If there is any user action needed during the infusion process, the dashboard may be used to prompt necessary actions from the user and give them visibility to what happens next. EHR data may be integrated and accessed on the infusion dashboard. If a patient has lab results that gait a certain portion of the infusion process these can be checked and displayed for the user.

The user may share their progress through the digital caregiver to keep their friends, family or other caregivers updated. These secondary users may opt-in to receiving notifications and updated in whatever manner works best for them. They may also receive notifications through text or email without needing their own account. With more complex medication regimens the different medications could be assigned different colors to differentiate them more visually.

A parallel dashboard view may be used for a caregiver with adapted content and language to fit the context of the caregiver. A nurse would see different wording than the patient's parent who is not a healthcare professional. For a professional nurse that has multiple patients with infusions, a dedicated dashboard may be used to manage data from multiple patients simultaneously. A help button may be used to link a patient to their designated caregiver during their injection. This communication may be in the form of chat or video. A designated caregiver or nurse may check in periodically throughout the infusion to make sure the patient is doing well. Offering support during an at-home infusion provides more structure for a patient that is on their own.

If the user goes to another software application on their TV, they can receive notifications on the progress of their infusion and quickly view the pop-up notification on their TV without disrupting what they are viewing. The user may ask how much time is left using an integrated voice assistant at any time to determine what their infusion progress is without closing the content they are viewing.

Referring to FIG. 7B illustrating the Smartwatch interface of the software application, the user can initiate contact with the digital caregiver by utilizing native voice functionality 309 that is integrated with the software application or by using another voice interface. The user may receive relevant feedback on the progress of their infusion or any other content that they request 310 and have the option to engage further with the software application or continue with their activities 311.

For users that are mobile during the infusion and want to stay up to date when not near the TV, they may be able to view the infusion progress using the digital caregiver Smartwatch interface 312 including the time remaining. They may be able to scroll to view more context on which medications have been administered 313. Once the infusion or a portion of the infusion is complete, they may receive a notification on their smartwatch 314. If there are any steps that need to be taken the user can be shown an abbreviated version on the Smartwatch or they can be redirected back to the TV interface to view the full information.

### Embodiment 5: Emergency Responder Mode

Patients may be on their own in their homes with these complex regimens and long infusions. Many of these medications may have the risk of an infusion reaction or other negative side effects. Patients need guidance and reassurance throughout the infusion process, and if something does go wrong, they need the proper action to be taken.

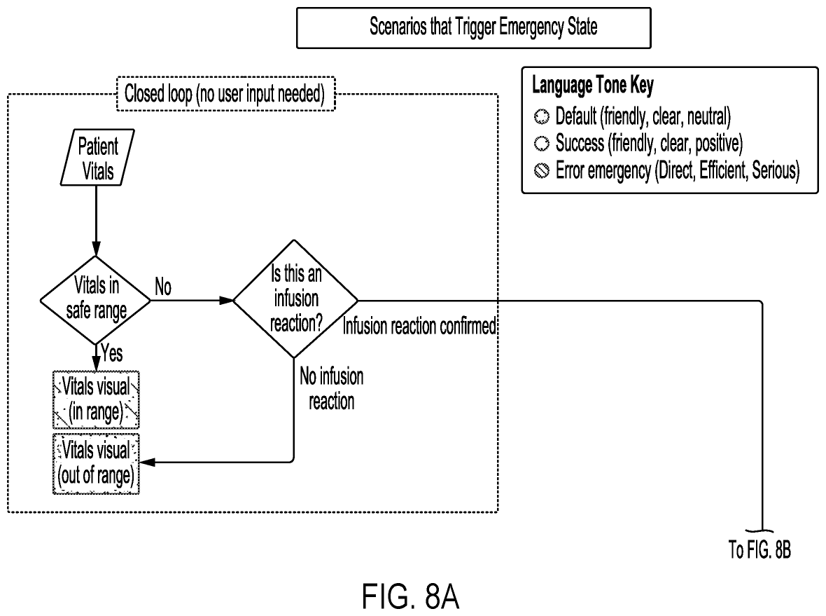

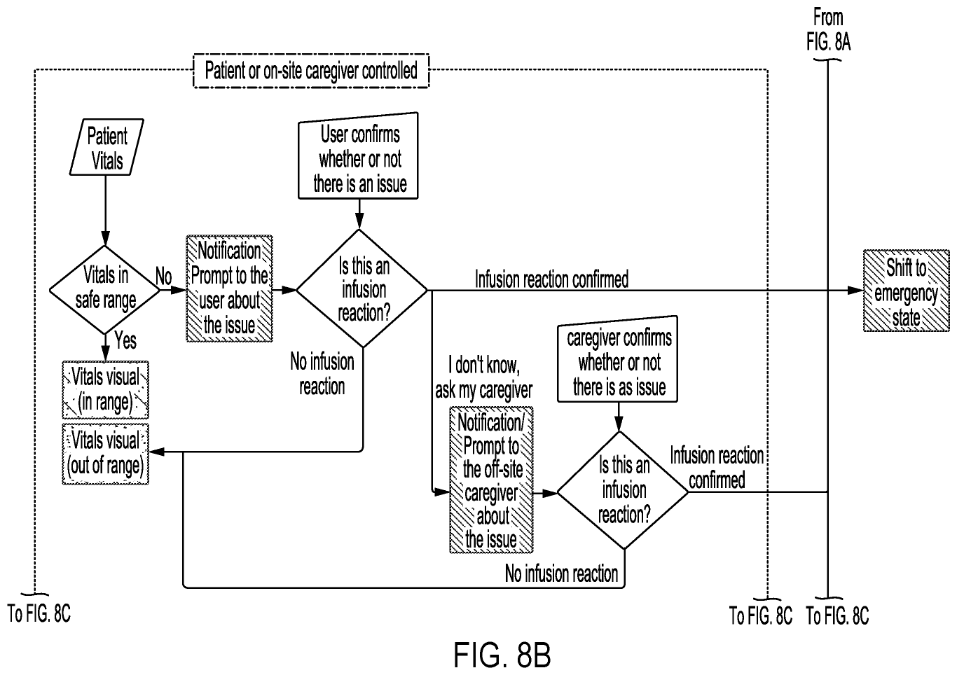

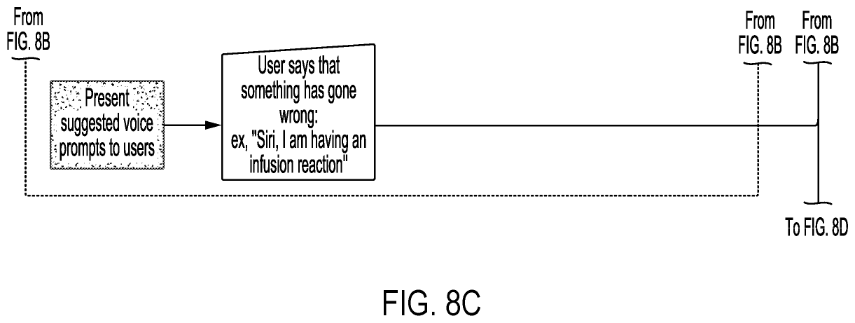

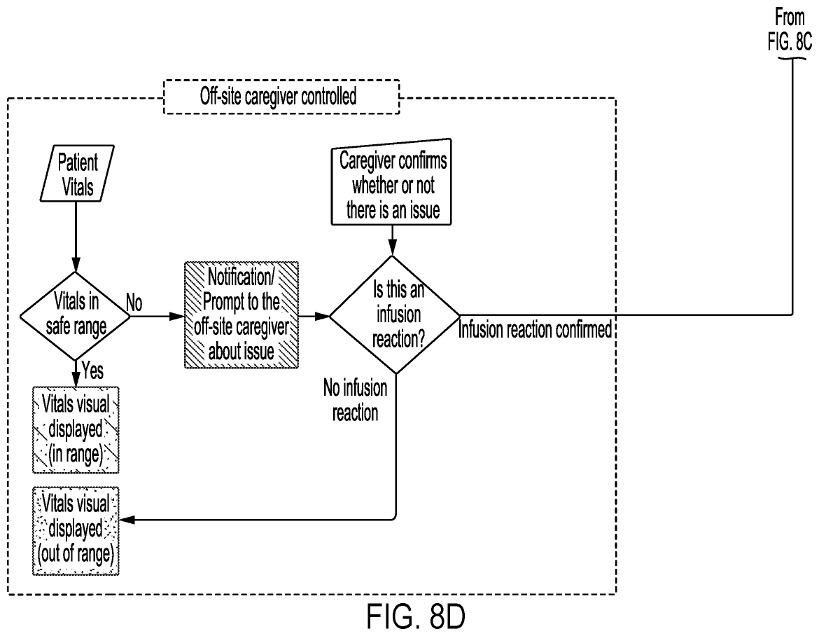

When patients have an injection reaction that requires intervention from emergency responders, a caregiver, or bystanders it is vital that the people assisting them are informed of the situation accurately and efficiently. Digital caregiver can use a deliberate shift in language in an emergency to communicate vital information more efficiently to the emergency responders and anyone else assisting the patient. FIGS. 8A-8D illustrate the different triggers for the emergency state.

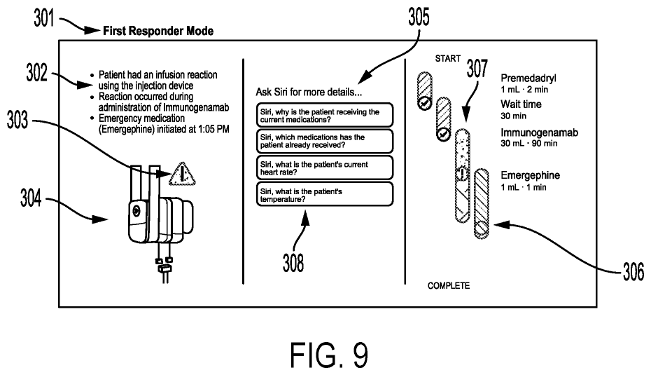

Referring to FIG. 9 illustrating an example dashboard view during an infusion reaction, the color scheme of the screen may shift to signify to the user that something has changed with the infusion 301. The most relevant medical information for an infusion reaction 302 may be presented in the top left as the content that the emergency responder or other user should read first. The information may be presented in a short and succinct way in a tone that reflects the severity of the situation. While the tone of language may shift, the content must still be easy to read and understand since the medical literacy of the person caring for the patient in an emergency situation cannot be assumed. Content should be below a chosen reading level and require minimal health literacy to interpret it correctly.

The visual representation of the device 304 can serve as an immediate context to communicate that the situation has to do with this specific device. Many people coming into the patient's house may have no idea what their infusion device is, and without a visual reference, they may not connect the device to the TV view. A warning or error state symbol 303 may be used on the visual to signify that something has gone wrong. This may be adapted depending on whether the emergency was caused by a malfunction with the device or an infusion reaction from the patient.

Any emergency responders or other second parties entering the patient's home will likely not be familiar with the digital caregiver interface. The voice prompts 305 may be presented again with a focus more on interrogating the device to determine what happened with the infusion. These voice prompts alert the emergency responder that they can use the voice assistant to learn more about the situation, and it outlines the structure of question 308 that can be answered.

The visual representation of the infusion timeline will shift into an emergency view which may have an error 307 shown for the most recent treatment medication that was administered. There may be a different style used for the emergency medication 306 to help that content stand out when users glance at the infusion timeline.

The style of the voice interface may change depending on the state of the injection. For example, if the patient is asking troubleshooting questions during setup the voice interface may use slow and thorough explanations of the actions that need to be taken. This contrasts with how the voice interface may handle the emergency scenario and speak to an emergency responder where it can switch to a more clinical and efficient communication style with shorter, more direct terminology and tone.

There are multiple methods for triggering the emergency scenario that is either automated or user-driven. If the digital caregiver has access to the vitals data from the patient's Smartwatch or other devices to determine if there is an infusion reaction happening and act. The dashboard may contain voice prompts that ask about the patient's current state with questions about how they feel to interrogate them and determine if they may be having an infusion reaction. These voice prompts may look like “I'm feeling itchy” or other similarly structured health questions.

If the digital caregiver does detect a possible or confirmed infusion reaction the patient must be alerted if they have not already noticed the issue. A notification with haptic feedback to the smartwatch or other device may be used to alert the user that they need to either confirm if they are having a reaction or to inform them that they are having a reaction. The digital caregiver may also be set up to call emergency services. This is particularly helpful if the patient cannot act due to their injection reaction symptoms. When an infusion reaction is detected and the patient takes no action when notified, this could be utilized as a safeguard to trigger a call for medical assistance.

Information about the emergency state of the patient may be communicated through multiple channels. Vital information may be shared in a form that is compatible with an existing emergency responder software in order to facilitate faster communication and even communicate with emergency services before they arrive. Existing patient care reports (PCRs) are used by emergency medical services to communicate patient information to the hospital. Facilitating data transfer to auto-generate medical history portions of the PCR may improve care at the hospital.

To accommodate any incompatibility with existing emergency response reporting systems, patient information may be provided in the form of something like a QR code, so that emergency responders can scan and use the data as needed. After downloading the relevant data an automated summary could be uploaded to the PCR or forwarded to hospital staff or other clinicians.

In one example, the present disclosure provides a method of providing visual feedback during a medicament injection to a patient. The method includes receiving, via a first computing device, information regarding the medicament injection. The method also includes displaying, via a display of a second computing device, a name of the medicament and a dosage amount of the medicament for the medicament injection. The method also includes displaying, via the display of the second computing device, one or more setup steps for preparing the medicament injection. The method also includes displaying, via the display of the second computing device, an injection dashboard providing one or more indications of a progress of the medicament injection. In one example, the first computing device comprises a smartphone and the second computing device comprises an Internet-connected television.

In one example, the information regarding the medicament injection is received manually via a patient or a caregiver at the first computing device. In another example, the information regarding the medicament injection is received by the first computing device scanning a QR code on a component of the medicament injection

In one example, the method further includes, (a) in response to receiving information regarding the medicament injection, comparing the received information to a patient profile, wherein the patient profile includes a list of one or more prescriptions associated with the patient, (b) if the information received regarding the medicament injection is consistent with the patient profile, proceeding to display, via the display of the second computing device, the one or more setup steps for preparing the medicament injection, and (c) if the information received regarding the medicament injection is not consistent with the patient profile, proceeding to display, via the display of the second computing device, an error message.

In one example, the one or more setup steps for preparing the medicament injection include a visual representation of one or more components for the medicament injection. In another example, the injection dashboard includes one or more of an injection timer, an indication of a completion time of the medicament injection, one or more voice prompts, one or more patient vitals, and a progress bar.

In one example, the method further includes displaying, via the display of the second computing device, an indication of one or more patient vitals during the medicament injection. In one example, the one or more patient vitals are measured via a third computing device, and wherein the measured one or more patient vitals are transmitted from the third computing device to the second computing device. In one example, the third computing device comprises a wearable device. In one example, the one or more patient vitals comprise one or more of a heartrate of the patient, a body temperature of the patient, a respiratory rate of the patient, a blood oxygen level of the patient, or a blood pressure of the patient.

In one example, the method further includes determining that at least one of the one or more patient vitals are outside of a predetermined acceptable range. In one such example, the method further includes (a) displaying, via the display of the second computing device, an indication that at least one of the one or more patient vitals are outside of the predetermined acceptable range, and (b) simultaneously calling an emergency services. In another exaple, the method further includes (a) displaying, via the display of the second computing device, an indication that at least one of the one or more patient vitals are outside of the predetermined acceptable range, and (b) in response to a determination that no action is taken by the patient, calling an emergency services. In another example, the method further includes (a) displaying, via the display of the second computing device, an indication that at least one of the one or more patient vitals are outside of the predetermined acceptable range, and (b) providing one or more voice prompts related to the indication that at least one of the one or more patient vitals are outside of the predetermined acceptable range.

In one example, the method further includes (a) displaying, via the display of the second computing device, a first set of one or more voice prompts if each of the one of the one or more patient vitals are within of a predetermined acceptable range, and (b) displaying, via the display of the second computing device, a second set of one or more voice prompts if at least one of the one of the one or more patient vitals are outside of a predetermined acceptable range, wherein the first set of one or more voice prompts are different than the second set of one or more voice prompts.

It will be appreciated that other arrangements are possible as well, including some arrangements that involve more or fewer steps than those described above, or steps in a different order than those described above.

While various aspects and embodiments have been disclosed herein, other aspects and embodiments will be apparent to those skilled in the art. All embodiments within and between different aspects of the devices and methods can be combined unless the context clearly dictates otherwise. The various aspects and embodiments disclosed herein are for purposes of illustration and are not intended to be limiting, with the true scope and spirit being indicated by the claims.

What is claimed is:

1. A method of providing visual feedback during a medicament injection to a patient, the method comprising:
receiving, via a first computing device, information regarding the medicament injection;
displaying, via a display of a second computing device, a name of the medicament and a dosage amount of the medicament for the medicament injection;
displaying, via the display of the second computing device, one or more setup steps for preparing the medicament injection; and
displaying, via the display of the second computing device, an injection dashboard providing one or more indications of a progress of the medicament injection.

2. The method of claim 1, wherein the information regarding the medicament injection is received manually via a patient or a caregiver at the first computing device.

3. The method of claim 1, wherein the information regarding the medicament injection is received by the first computing device scanning a QR code on a component of the medicament injection.

4. The method of claim 1, further comprising:
in response to receiving information regarding the medicament injection, comparing the received information to a patient profile, wherein the patient profile includes a list of one or more prescriptions associated with the patient;
if the information received regarding the medicament injection is consistent with the patient profile, proceeding to display, via the display of the second computing device, the one or more setup steps for preparing the medicament injection; and
if the information received regarding the medicament injection is not consistent with the patient profile, proceeding to display, via the display of the second computing device, an error message.

5. The method of claim 1, wherein the one or more setup steps for preparing the medicament injection include a visual representation of one or more components for the medicament injection.

6. The method of claim 1, wherein the injection dashboard includes one or more of an injection timer, an indication of a completion time of the medicament injection, one or more voice prompts, one or more patient vitals, and a progress bar.

7. The method of claim 1, further comprising:
displaying, via the display of the second computing device, an indication of one or more patient vitals during the medicament injection.

8. The method of claim 7, wherein the one or more patient vitals are measured via a third computing device, and wherein the measured one or more patient vitals are transmitted from the third computing device to the second computing device.

9. The method of claim 8, wherein the third computing device comprises a wearable device.

10. The method of claim 7, wherein the one or more patient vitals comprise one or more of a heartrate of the patient, a body temperature of the patient, a respiratory rate of the patient, a blood oxygen level of the patient, or a blood pressure of the patient.

11. The method of claim 7, further comprising:
determining that at least one of the one or more patient vitals are outside of a predetermined acceptable range.

12. The method of claim 11, further comprising:
displaying, via the display of the second computing device, an indication that at least one of the one or more patient vitals are outside of the predetermined acceptable range; and
simultaneously calling an emergency services.

13. The method of claim 11, further comprising:
displaying, via the display of the second computing device, an indication that at least one of the one or more patient vitals are outside of the predetermined acceptable range; and
in response to a determination that no action is taken by the patient, calling an emergency services.

14. The method of claim 11, further comprising:
displaying, via the display of the second computing device, an indication that at least one of the one or more patient vitals are outside of the predetermined acceptable range; and
providing one or more voice prompts related to the indication that at least one of the one or more patient vitals are outside of the predetermined acceptable range.

15. The method of claim 7, further comprising:
displaying, via the display of the second computing device, a first set of one or more voice prompts if each of the one of the one or more patient vitals are within of a predetermined acceptable range; and
displaying, via the display of the second computing device, a second set of one or more voice prompts if at least one of the one of the one or more patient vitals are outside of a predetermined acceptable range, wherein the first set of one or more voice prompts are different than the second set of one or more voice prompts.

16. The method of claim 1, wherein the first computing device comprises a smartphone.

17. The method of claim 1, wherein the second computing device comprises an Internet-connected television.
